(19) United States
(12) Patent Application Publication
(10) Pub. No.: US 2017/0101619 A1
(43) Pub. Date: Apr. 13, 2017

(54) PHOTOBIOREACTOR IN A CLOSED ENVIRONMENT FOR CULTIVATING PHOTOSYNTHETIC MICRO-ORGANISMS

(71) Applicant: ACTA ALGA, Paris (FR)

(72) Inventors: Jacques Bourgoin, Thomery (FR); Michel Conin, Paris (FR); Alain Friederich, Paris (FR); Guocai Sun, Bourg La Reine (FR)

(21) Appl. No.: 15/385,478

(22) Filed: Dec. 20, 2016

Related U.S. Application Data
(63) Continuation of application No. 13/520,330, filed on Oct. 2, 2012, now abandoned.

(30) Foreign Application Priority Data
Jan. 4, 2010 (FR) 1050015

(51) Int. Cl. C12M 1/00; C12M 1/34

(52) U.S. Cl. CPC C12M 43/08; C12M 21/02; C12M 31/08; C12M 41/12; C12M 23/22

(57) ABSTRACT

The invention relates to a photobioreactor for cultivating photosynthetic micro-organisms, comprising: a) at least one cultivation container (1) for containing the culture medium (3) of the micro-organisms, b) photovoltaic cells (2) isolated from the culture medium (3), emitting light towards the culture medium (3), and c) means (4) for powering the photovoltaic cells (2) in order to operate the photovoltaic cells in light emission mode.

The invention relates to intensive continuous cultivation of photosynthetic micro-organisms.

Microalgae are photosynthetic plant organisms wherein the metabolism and growth require, among other things, CO₂, light and nutrients.

Numerous applications of the industrial cultivation of microalgae are known.

Microalgae can be cultivated to reuse and purify carbon dioxide, NOx and/or SOx emissions from some industrial plants (WO 2008042919).

The oil extracted from microalgae can be used as a biofuel (WO2008070281, WO2008055190, WO2008060571).

Microalgae may be cultivated for the production of omega-3 and polyunsaturated fatty acids thereof.

Microalgae may also be cultivated to produce pigments.

The large-scale industrial cultivation of microalgae uses the sun as a light source. For this purpose, the microalgae are frequently placed in open tanks (“raceways”) with or without circulation (US2008178739). Tubular or plate photobioreactors are also found, consisting of translucent materials, enabling the passage of light rays in the culture medium and wherein the microalgae circulate (FR26213223). Further three-dimensional transparent tube network systems can improve the use of the space (EP0874043).

These installations are extremely large and the production yields are low given the uncertainties in respect of sunlight and night phases having adverse effects on microalga growth.

In order to reduce the size and enhance the efficiency, closed photobioreactors have been developed. They use the availability of artificial lighting 24 hours a day and 7 days a week, with the option of switching off the lighting according to the specific sequences of the biological cycles of the algae involved.

Indeed, the crucial factor in increasing the biomass of microalgae is light, both in terms of quantity and quality since microalgae only absorb certain white light wavelengths.

A photobioreactor is defined as an enclosed system wherein biological interactions take place, in the presence of light energy, to be controlled by controlling the cultivation conditions.

The more suitable to the light dispensed in the photobioreactor to the microalga species, the more advantageous the biomass production.

A first artificial lighting solution for solving this problem consists of conveying the light from a light source in the culture medium in the vicinity of the microalgae using optical fibres (U.S. Pat. No. 6,156,561 and EP0935991).

The optical fibres may be associated with further immersed means guiding the light inside the container (JP2001178443 and DE29819259).

The major drawback is that this solution is only suitable for obtaining low (light produced)/(effective light) yields. Indeed, the intensity is reduced due to the interfaces between the light sources and the waveguide and it is difficult to couple more than one light source on the same fibre. Moreover, a problem arises once a plurality of different wavelengths is used: indeed, to extract light from the optical fibres immersed in the culture medium, it is necessary to perform a surface treatment (roughness), to diffuse or diffract a fraction of the light guided. The most efficient solution consists of etching a grid on the periphery of the fibre with spacing in the region of the wavelength of the light carried. This solution has a narrow bandwidth and is completely unsuitable when a plurality of wavelengths is used. The use of random roughness is low efficiency.

A further artificial lighting solution for solving this problem consists of immersing light sources directly in the photobioreactor container, such as for example fluorescent lamps (U.S. Pat. No. 5,104,803) or LEDs (Light-Emitting Diodes) (DE202007013406 and WO2007047805).

This solution makes it possible to enhance the energy efficiency of the lighting method since the light sources are closer to and coupled better with the culture medium.

However, the use of light sources introduced into the reactor, particularly LEDs, needs to account for three further major problems.

The first is inherent to the penetration of light into the culture, which is directly linked with the density of the microalgae. This density increases during the cultivation process and rapidly leads to the light output being extinguished in most of the reactor.

Solutions consisting of illuminating the inner wall of the photobioreactor (DE202007013406) thus cannot be transposed to industrial scale photobioreactors of several hundreds of litres merely by homothetic transformation, the light absorption lengths still being centimetric at the end of the breeding process.

To remove the shaded areas appearing during the cultivation process, it is possible to multiply the light sources in the container and position them sufficiently close to each other to illuminate the culture medium regardless of the variable absorption lengths associated with the biological cycle. Doing so poses the problem of managing the heat of the reactor which needs to be controlled within a few degrees, and which is dependent on the type of algae. This heat management is the second major problem to be solved. It is inherent to these first-generation reactor structures, regardless of the type of light sources used. There is an additional problem of the cost of the photobioreactor if the light sources need to be multiplied in a large number.

The third problem is that of obtaining a homogeneous illumination front in terms of intensity in the reactor growth volume. In addition to the progressive decline in the light wave intensity by absorption in the culture medium, significant light energy dispersion on the incident light front takes place. This impedes the optimisation of the biomass growth method for a given overall incident light energy.

In order to address these problems, the inventors discovered, unexpectedly and surprisingly, a novel light source suitable for photobioreactors: photovoltaic cells used in direct injection emitting light under these conditions.

This light source offers the advantages of being particularly homogeneous and being suitable for being optimised for the alga strain to be produced since the photovoltaic cells can be adapted to emit the wavelength(s) absorbed by the strain for the photosynthesis thereof.

Consequently, the subject-matter of the invention is that of a photobioreactor for cultivating photosynthetic micro-organisms, preferably microalgae, comprising:
(a) at least a culture enclosure (1) for containing the culture medium (3) of the micro-organisms,
(b) photovoltaic cells (2) isolated from the culture medium (3) emitting light to the culture medium (3)
(c) means (4) for powering the photovoltaic cells (2) in order to operate the photovoltaic cells in light emission mode.
   A photovoltaic cell is an electronic component which, when exposed to light (photons), generates electricity. The most common photovoltaic cells consist of semiconductor materials. In order to obtain light emission, these semiconductor materials need to be with a direct gap, such as alloys of As, Ga, In, P. The silicon (Si) material is unsuitable for this function as the gap thereof is indirect. They are generally in the form of thin panels measuring some ten centimetres on the side, sandwiched between two metal contacts, for a thickness in the region of one millimetre. The principle of photovoltaic cells is well known (Physics of Semiconductor Devices-J Wiley & Sons, 3rd Edition, Simon M. Sze, Kwok. Ng).

In the semiconductor exposed to light, a photon of sufficient energy extracts an electron, thus creating a “gap”. Normally, the electron quickly finds a gap to reposition itself, and the energy supplied by the photon is thus dissipated. The principle of a photovoltaic cell is that of forcing the electrons and the gaps to each move towards an opposite face of the material rather than merely recombining therein: in this way, a difference in potential and thus a voltage between the two faces will appear, like a battery.

For this, it is necessary to create a permanent electrical field by means of a PN junction, respectively between two P and N-doped layers. In the top layer of the cell, there is a greater quantity of free electrons than a layer of pure material, hence the term N doping, for negative (electron charge). In the bottom layer of the cell, the quantity of free electrons is less than a layer of pure materials, the electrons are bound to the crystalline network which, as a result, is positively charged. Electricity is conducted by positive gaps (P).

When the P-N junction is created, the free electrons in the N region enter the P layer and are recombined with the gaps in the P region. In this way, for the lifetime of the junction, there will be a positive charge of the N region at the edge of the junction (because the electrons have left) and a negative charge in the P region at the edge of the junction (because the gaps have disappeared) and there is an electric field between the two, from N to P.

In conventional operation, a photon extracts an electron from the matrix, creating a free electron and a gap. The electrons accumulate in the N region (which becomes the negative pole), whereas the gaps accumulate in the P doped layer (which becomes the positive pole).

Cells having a high efficiency have been developed for space applications: multi-junction cells consisting of a plurality of thin layers, conventionally of one to five junctions.

A triple-junction cell, for example, consists of the semiconductors AsGa, Ge and GaInP2. Each type of semiconductor is characterised by a maximum wavelength above which it is incapable of converting the photon into electrical energy. Below this wavelength, the excess energy carried by the photon is lost.

According to the present invention, the photovoltaic cells are used in reverse emission mode, i.e. as a light source. They are powered with an electric current called an “injection current” and unlike the conventional operation thereof described above produce light. If a positive voltage is applied at the P region end, the main positive carriers (the gaps) are pushed towards the junction. At the same time, the main negative carriers at the N end (the electrons) are attracted to the junction. Once they reach the junction, the carriers are recombined, releasing photons having energies corresponding to the gaps of the semiconductor materials used. Fundamentally, a photovoltaic cell used in direct injection is a large-area light-emitting diode. Furthermore, it differs from a LED by the geometry of the injection contacts thereof which need to cover a large surface area. Conventionally, contact grids are created with fingers spaced by a length less than the carrier diffusion length. This large-area LED can benefit from all the internal and external quantum yield enhancements implemented in conventional LEDs (Bragg reflector, use of quantum wells in the active layer, surface treatments, etc.). Indeed, to come out of the device, the photons need to pass through (without being absorbed by) the semiconductor, from the junction to the surface, and pass through the surface of the semiconductor without being subject to reflection and, in particular, not be subject to the total internal reflection which returns the photons to inside the cell where they are eventually absorbed. Those which are not subject to total internal reflection leave the semiconductor and form the external optical flow (to the air, for example).

In point LEDs, the external transfer efficiency is enhanced marginally by introducing optics bonded on the surface of the semiconductor (intermediate optical index between that of air (n=1) and that of the semiconductor (3<n<4)). Under these conditions, the best LEDs have external quantum yields of approximately 20% (external light power over electrical power supplied to the component). For a larger flat LED according to the invention, the solution would be that of microstructuring the surface so as to increase the probability of the photon encountering a surface in a quasi-perpendicular fashion. The highest external quantum yield ever obtained to date is slightly greater than 45%. Various microstructuring methods are currently the subject of laboratory studies and are based on micronic lithography techniques in use in the semiconductor industry, or on techniques for etching the external surface of the LED. In the latter technology category, external quantum yields in the region of 30% are routinely obtained. Using large-area components makes the application of these technologies much easier.

The photovoltaic cells used according to the present invention are made of a direct gap material (AsGa, GaInP, etc.). In these materials, the energy released during the recombination of a gap-electron pair is conveyed by the emission of an optionally visible photon. The light intensity is directly proportional to the injection current. The light emission wavelength is equivalent to the gap energy of the semiconductor material forming the photovoltaic cell. An indirect gap semiconductor material does not emit light, the energy being dissipated in the form of heat. Conventionally, the direct gap materials emitting in the visible range are III/IV or II/VI alloys.

The light emitted consists of direct radiative transitions of the constituent materials of the photovoltaic cell. In this way, it is possible to choose a photovoltaic cell made of one or a plurality of materials emitting in one or a plurality of wavelengths, advantageously the wavelength(s) of the photosynthetic micro-organism species to be cultivated in the photobioreactor according to the invention.

Preferentially, the photovoltaic cells used in the present invention are cells with one, two or three junctions.

Preferentially, the substrate thereof is germanium or AsGa which have comparable network parameters to those of the materials to be grown epitaxially to produce the junctions. The use of silicon as a substrate requires, as demonstrated in the literature, the use of Smart-Cut® technology, which consists of separating the active part of the component (produced on a layer of AsGa or Germanium) and bonding same by molecular adhesion onto the silicon substrate.

Preferentially, the direct gap materials covering the substrate are III/IV alloys according to the periodical table of the elements, particularly preferably AsGa (Arsenic-Gallium), GaInP (Gallium-Indium-Phosphorus) and/or GaInAs (Gallium-Indium-Arsenic), although any direct gap materials are suitable.

Particularly preferably, the photovoltaic cells (2) used in the present invention are cells made of AsGa and/GaInP material on a germanium substrate.

The materials are chosen according to the emitting wavelength thereof. Indeed, one of the advantages of the photobioreactor according to the invention is that of supplying the cultivated photosynthetic micro-organism with the specific wavelength(s) absorbed for the photosynthesis thereof and thus optimising the biomass multiplication conditions.

Advantageously, the photovoltaic cells used according to the invention have a substrate and one or two direct gap materials, i.e. two or three junctions, and emit at one or two wavelengths. Advantageously, they emit at wavelengths corresponding to chlorophyll pigments. Advantageously, they emit at wavelengths within the intervals of 400 to 450 nm and 640 to 700 nm.

The photovoltaic cells measure some tens of square centimetres, conventionally approximately 100 cm². According to the present invention, they are preferably arranged on panels (7). Particularly preferably, they cover panels (7) to form, by juxtaposition, a plane homogeneous lighting system up to a surface area in the region of one square metre. They may consist of various materials on either side of the panel. For example, one side of the panel may be covered with photovoltaic cells emitting one wavelength and the other side of the panel may be covered with photovoltaic cells emitting another wavelength.

The photovoltaic cells (2) are preferably placed:
either in the culture enclosure(s) (1) in sealed containers (5) of adapted transparency (TA) immersed in the culture medium (3).
or outside the culture enclosure(s), at a short distance from the external wall of the culture enclosure(s) (6), said wall consisting of a material of adapted transparency for the passage of the wavelength(s) emitted. In one particular embodiment, the photobioreactor according to the invention comprises a plurality of culture enclosures separated by photovoltaic cells. For example, a plurality of parallelepipedic culture enclosures, for example two, is stacked and separated by panels (7) of photovoltaic cells (2) (see FIGS. 6).

The containers of “adapted transparency” (TA) are containers providing an optimum optical yield in the wavelengths providing photosynthesis. The suitable adapted transparency materials are PMMA (polymethyl methacrylate), Plexiglas, glass, polycarbonate, PMMA panels.

The term “short distance” refers to a few millimetres to a few tens of centimetres, preferably from a few millimetres to a few centimetres.

In particular, it consists of a distance of 0.1 to 20 cm, preferably from 0.5 to 5 cm, more preferably from 0.5 to 2 cm, particularly preferably approximately 1 cm.

In a certain type of photobioreactor operation, it may be preferred to use a low microalga concentration. As a result, a significant fraction of the light is either not absorbed by the culture medium and thus leaves the reactor. This light may be returned to the culture medium for a second passage, by converting the external wall of the reactor into a mirror (e.g. Al, Ag metal coating).

The culture enclosure (1) conventionally has a cylindrical or parallelepipedic shape.

The photovoltaic cells are powered via injection contacts (8). These contacts (8) are preferably arranged at the end of the panels of emitting photovoltaic cells (2). They have a low resistivity, referred to as ohmic. Modulating the spacing of the contact grid enables the spatial modulation of the front of the light energy emitted.

Preferentially, the photovoltaic cells are assembled in a serial architecture.

Advantageously, the photovoltaic cells are electrically insulated from the substrate thereof by an insulator having a good thermal conductivity, for example of the Mylar® type, a polyethylene terephthalate developed by DuPont de Nemours.

According to one embodiment, the photobioreactor according to the invention comprises a system (8) for cooling the photovoltaic cells (2). Advantageously, the cooling system (9) consists of a heat transfer fluid (10) circulating in sealed containers (5), said containers (5) being connected to an external cooling device with respect to the sealed containers for the heat transfer fluid (10).

Advantageously, the heat transfer fluid (10) is chosen from the transparency thereof in the wavelength ranges from 0.3 microns to 1 micron and there should not be significant absorption in this wavelength range. Suitable heat transfer fluids are silicone oil, perfluorinated oil or air.

The heat transfer fluid (10) cools the photovoltaic cells (2) directly by contact. It is conveyed to and cooled by the cooling system of the photobioreactor according to the invention, external to the culture enclosure (1). The heat regulation of this fluid further enables thermostatic control of the culture enclosure.

The photobioreactor according to the invention may further comprise a system for injecting gas (11), particularly CO₂ into the culture enclosure (1).

The culture enclosure (1) of the photobioreactor according to the invention may be designed for varied industrial or laboratory applications.

The dimensions of a laboratory-scale culture enclosure (1) are from a few tens of centimetres to a few hundreds of centimetres for the height and diameter (cylindrical container) or width (parallelepipedic container). The volume of a laboratory-scale culture enclosure (1) is less than one m³. Advantageously, the culture enclosure (1) is an industrial culture enclosure (1).

The dimensions of an industrial-scale culture enclosure (1) are several metres.

The volume of an industrial-scale culture enclosure (1) is greater than one m³. The culture enclosure (1) is made of a suitable material for containing the culture medium, made of metal or polymer for example, and, preferentially selected from the group consisting of PMMA, polycarbonate or stainless steel. Containers made of a concrete type structural material for example may also be envisaged.

According to the embodiment wherein the photovoltaic cells are placed outside the culture enclosure, the culture enclosure is made of a material of adapted transparency.

According to the embodiment wherein the photovoltaic cells are placed in the culture enclosure, the internal walls (12) of the culture enclosure (1) of the photobioreactor are advantageously reflective so as to minimise light ray loss outside the closed container. They may be covered with a reflective material or paint. The energy expenditure required for the cultivation of the photosynthetic micro-organisms is thus reduced.

The photobioreactor according to the invention may further comprise a system (13) for mixing the culture medium (3).

The mixing system (13) has two main functions. Firstly, it needs to promote homogenisation of the temperature of the culture medium. Secondly, it enhances the homogenisation of the illumination of the micro-organisms. Indeed, by means of this mixing, the micro-organisms are moved from the areas with the most illumination to the areas with the least illumination and conversely.

The mixing of the culture medium is carried out by means of various techniques, the most common at the present time being referred to as the “air-lift” technique. Mechanical stirring may also be used: Archimedes screw, water propeller, Rushton type, hydrofoil, etc.

Advantageously, the mixing technique used is that referred to as “air lift” consisting of injecting a pressurised gas, for example air, into the lower part of the culture enclosure (1). The air, which has a lower density than the liquid, rises rapidly in the form of bubbles. The liquid and the microalgae are carried by the upward movement of the bubbles. The air may be injected vertically but also at an angle so as to cause liquid to be carried from one wall of the culture medium to the other, promoting mixing of the nutrients and CO₂ required by the microalgae. This movement of the culture liquid also ensures an average illumination to all the microalgae as they rise. The microalgae then fall back down into the volume where no air bubbles are rising. A closed culture liquid circuit is thus created. This technique enables mixing involving a low energy consumption and low stress for the microalgae.

The culture medium may be mixed partly by means of a conventional air-lift system, which essentially generates a vertical impulse, completed with an original lateral (CO₂+air) injection system distributed using feeders (14) along the height of the culture enclosure. The term “feeders” refers to a line or tube suitable for carrying gas or water from the source to the point at which the gas or water is to be injected. Said feeders (14) will be installed in the cultivation area against the walls (20) of the sealed containers (5) or the culture enclosure (1). The injection nozzles (15) are distributed on one (or more) feeder(s) (14). The number thereof and the inclination thereof will be dependent on the type of impulse to be transmitted to the micro-organisms (transverse impulse, vertical impulse, or impulse suitable for creating an overall movement of the biomass, enabling the algae to move periodically from one edge of the reactor to the other, with an upward movement). Advantageously, this ability to manage the transverse movement of the biomass will be used for homogenising the illumination thereof, i.e. preferentially directed upwards with a precise inclination. Furthermore, in this reactor design, it is possible to adapt the intensity of the transverse impulse such that the micro-organism transit time between the illuminated and non-illuminated areas spatially creates the illumination cycle required for the growth of some types of algae (illumination time/off time).

Advantageously, a volume of culture is regularly or continuous removed from the top part of the culture enclosure (1) and immediately replaced by the injection of an equivalent volume of water containing nutrients at the bottom part of the culture enclosure (1) or in the feeders (14). This method helps reduce the energy required to induce the circulation of the liquid in the reactor.

The cooling system (9) makes it possible to remove the heat released by the photovoltaic cells (2) while adjusting the temperature of the culture medium (3) of the photobioreactor.

The cooling system (9) may consist of a heat exchanger. For example, this heat exchanger consists of means for conveying (16) the hot heat transfer fluid (10) outside the culture enclosure (1), for example pipes connected to the upper end of the culture enclosure (1) coupled with a pump (17), and a cooler (18) consisting of circulating the hot heat transfer fluid in the opposite direction of cold water (see FIG. 8). Advantageously, the heat transfer fluid (10) is discharged from the culture container (1) at one of the ends thereof, at the top or at the bottom and enters the culture enclosure (1) via the other end. The cold heat transfer fluid (10) returns to the culture enclosure (1) via means for conveying same (19), for example pipes.

Advantageously, the number of photovoltaic cells in the photobioreactor according to the invention is such that they cover panels (7) extending approximately along the entire height of the culture enclosure (1).

The arrangement of the panels (7) of photovoltaic cells (2) is adapted to the shape of the photobioreactor.

For example, when the photobioreactor has a cylindrically shaped culture enclosure, the panels form a tube with a polygonal, preferably hexagonal or octagonal, cross-section together, for optimum approximation of the cylindrical shape (see FIG. 7).

In order to correct the edge effects in the corners of the polygon, the intensity of the injection current may be adapted locally since the light intensity is proportional to the intensity of the injection current. The intensity of the injection current may be adapted by modulating the spacing of the injection contact grid (8).

It is also possible to use a polymer diffusing material for enhancing the homogenisation of the wavefront. This thin layer material may cover the external walls (20) of the tight containers (5) if the panels (7) of photovoltaic cells are placed in the culture enclosure or the walls of the culture enclosure (6) if the panels (7) of photovoltaic cells (2) are placed outside the culture enclosure (1) at a short distance from the external walls (6).

A further aim of the invention is that of using photovoltaic cells (2) powered in reverse light mode for illuminating the culture medium of a photobioreactor.

A further aim of the invention is that of using a photobioreactor according to the invention for cultivating photosynthetic micro-organisms, preferably microalgae.

Further features and advantages of the invention will emerge more clearly on reading the description of the embodiments of the invention. The description refers to the following appended figures.

### FIGURES

FIG. 1: LED emission diagram

FIG. 2: Photovoltaic cell emission diagram

FIG. 3: Photovoltaic cell emission diagram with injection current boost at edges

FIG. 4: LED juxtaposition emission diagram

FIG. 5: Juxtaposed photovoltaic cell panel emission diagram

FIG. 6a-6b: Perspective and front view diagrams of a parallelepipedic photobioreactor comprising a panel of photovoltaic cells inserted between two culture enclosures

FIG. 7a-7b: Perspective and radial section diagrams of a cylindrical photobioreactor comprising a panel of photovoltaic cells arranged on a hexagonal cross-section tube placed in a sealed tube immersed in the culture medium.

FIG. 8: Presentation of the photovoltaic cell cooling system and the photobioreactor temperature regulation system

FIG. 9: Detailed diagram of the system for mixing the culture medium installed on a wall.

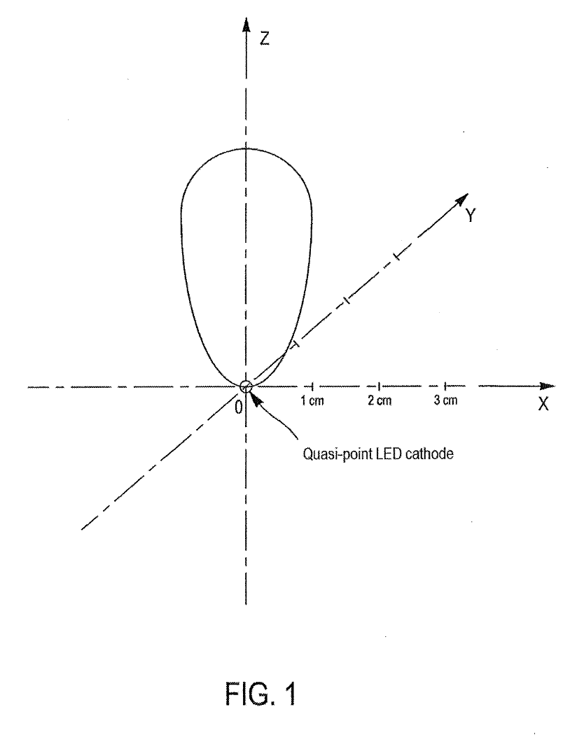

FIGS. 1 to 5 are energy emission diagrams. A quasi-point LED emits the energy thereof in “Lambertian” mode (lobe). Most of the energy is emitted perpendicular to the surface of the semiconductor. This energy decreases on moving away from the normal to the semiconductor. It is zero parallel with the surface thereof. Extending the emissive surface beyond the natural lobe width makes it possible, by adding the basic lobes, to create an energy-constant emissive surface in the planes parallel with the surface of the semiconductor (xOy). In the figures, the LED or photovoltaic cell is O-centred and the surface thereof is oriented perpendicular to (Oz). A section of these lobes is shown along the plane (xOz).

FIG. 1 represents the emission diagram for an LED situated at the centre of the reference. The cathode is assumed to be quasi-point (less than one mm² in size). There is invariance by rotating about the axis (Oz).

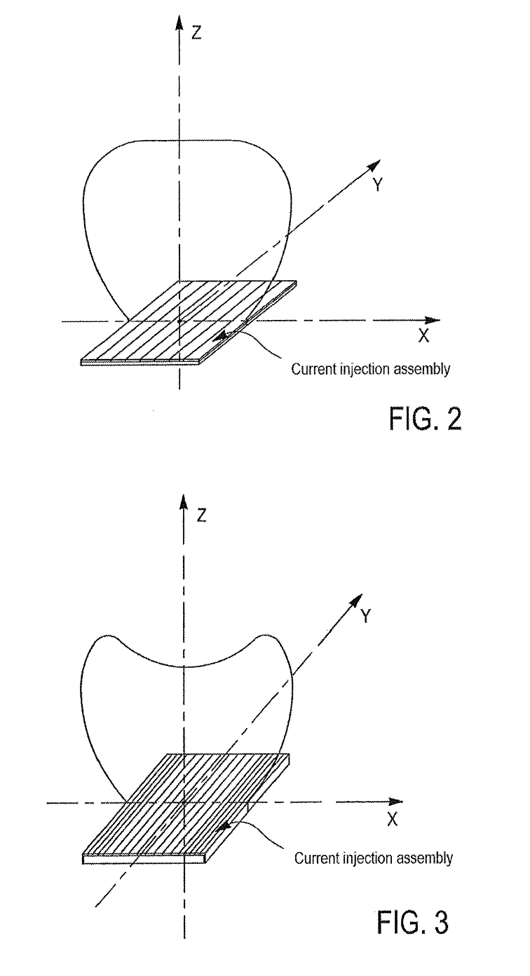

FIG. 2 represents the emission diagram for an inverted photovoltaic cell as used by the invention, in this case, with constant spacing of the current injection fingers. The light intensity in the plane parallel with (xOy) is constant in the vicinity of the centre of the cell.

FIG. 3 represents the energy emission diagram for an inverted photovoltaic cell when the spacing of the current injection fingers is retracted by moving the edges closer together. The injected current density is greater on the edges, hence the increase in light intensity.

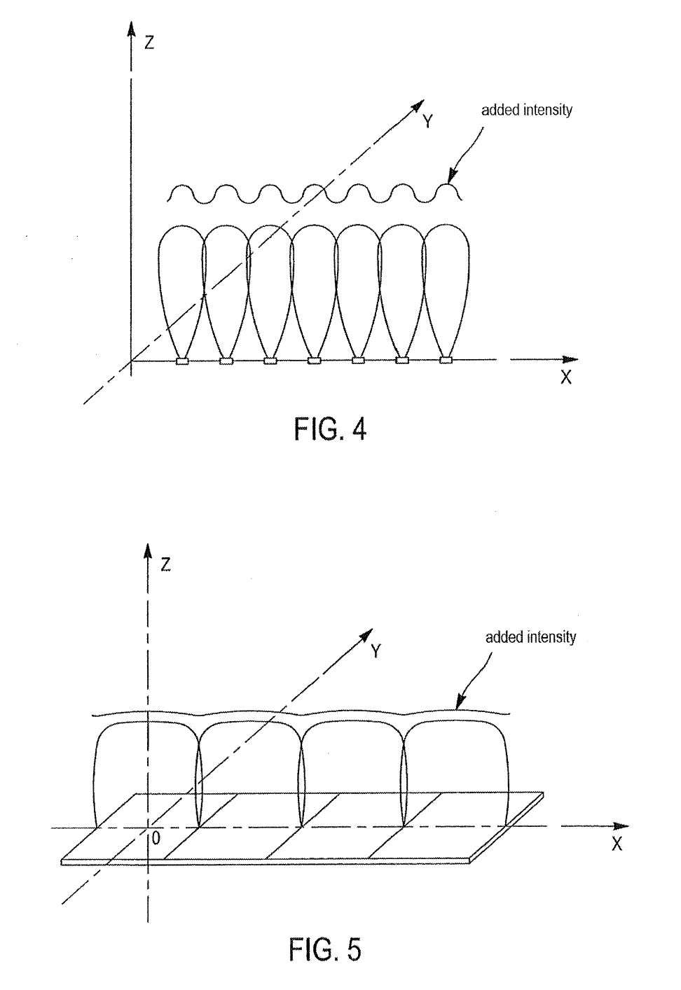

FIG. 4 represents the emission diagram of a strip of LEDs (arranged along (Ox)). The addition of the light outputs gives rise to an inhomogeneous front, the inhomogeneity whereof is dependent on the distance between two successive LEDs on the strip.

FIG. 5 represents the emission diagram of a strip of LEDs (arranged along (Ox)). If the cells are close enough, the light intensity in a plane parallel with (xOy) is constant), the energy received is thus only dependent on the distance to the cell: indeed, the output inhomogeneity is independent of the distance at which the measurement is made.

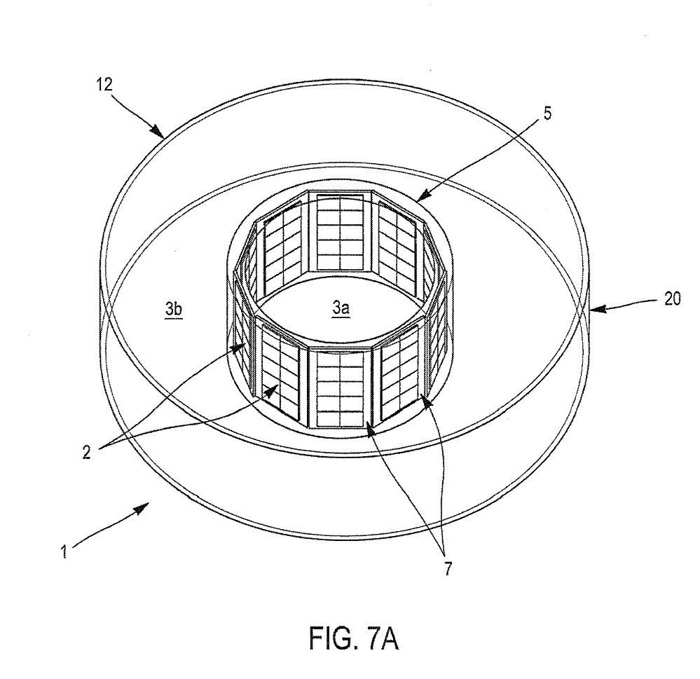

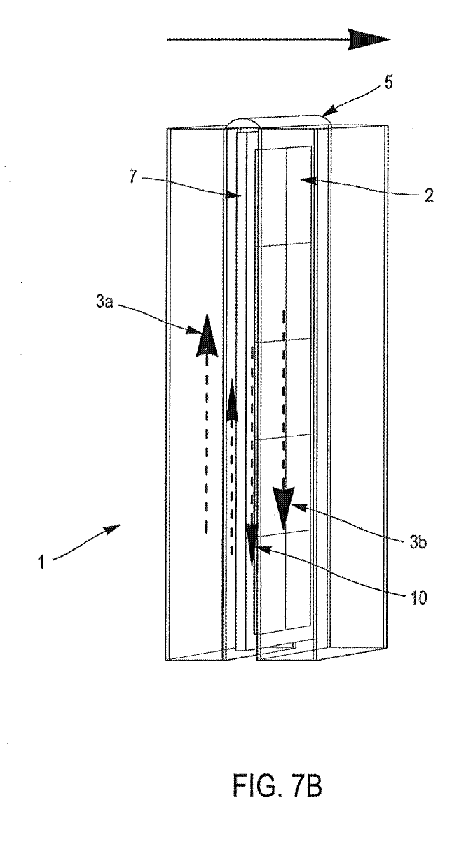

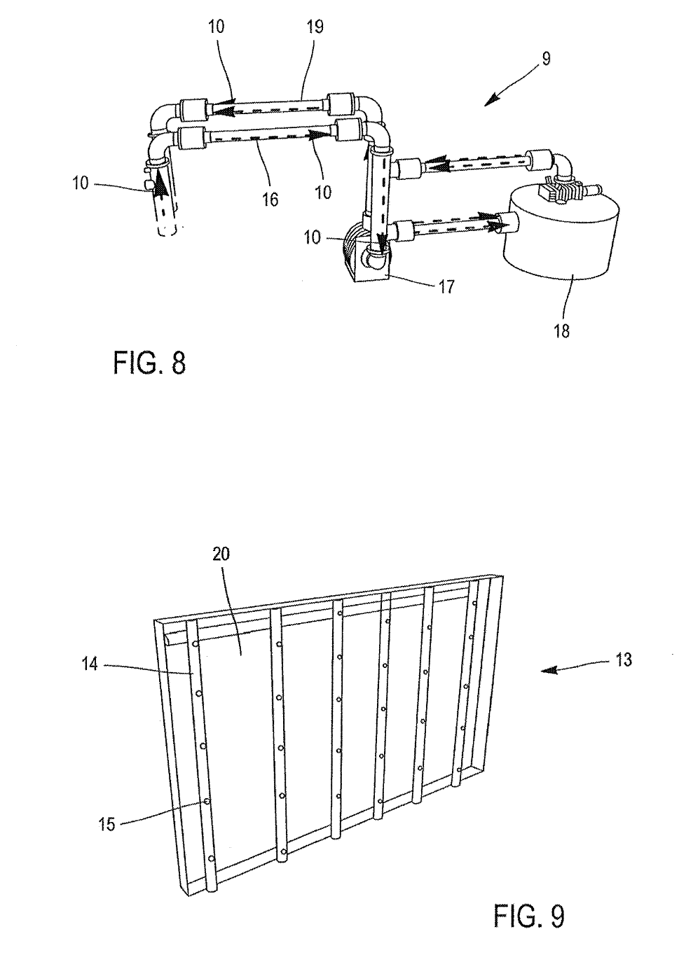

According to a first embodiment, the photobioreactor is cylindrical (FIGS. 7). Photovoltaic cells (2) are arranged on both faces of six panels (7) forming a tube having a hexagonal cross-section together. The length of these panels (7) is the height of the photobioreactor. These panels (7) are placed in a sealed tube (5) made of light-transparent material (glass, plastic, etc.), in turn immersed in the culture medium (3), separating same into an “internal” par (3a) and an “external” part (3b), seen in FIG. 7a. The panels are connected to current injection contacts (8).

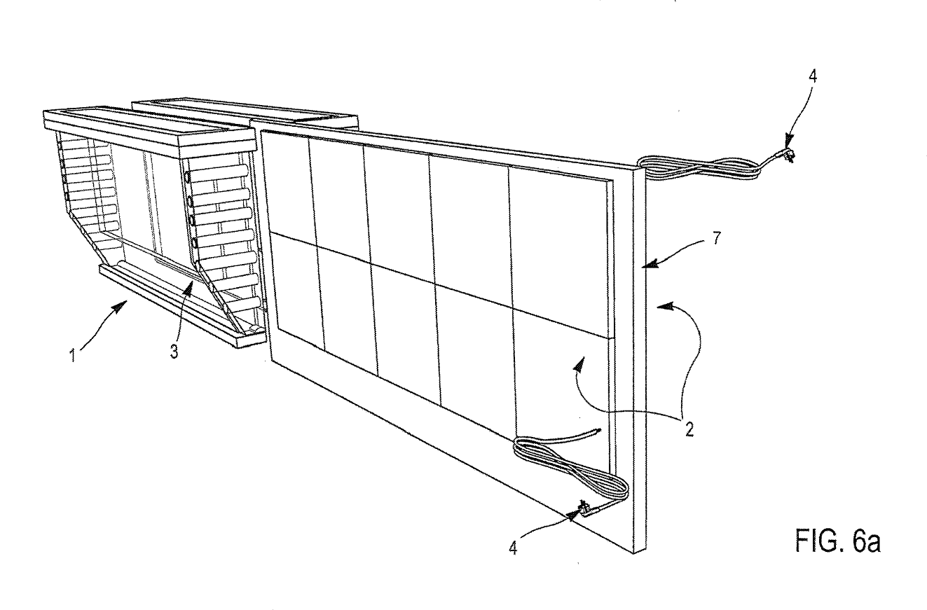

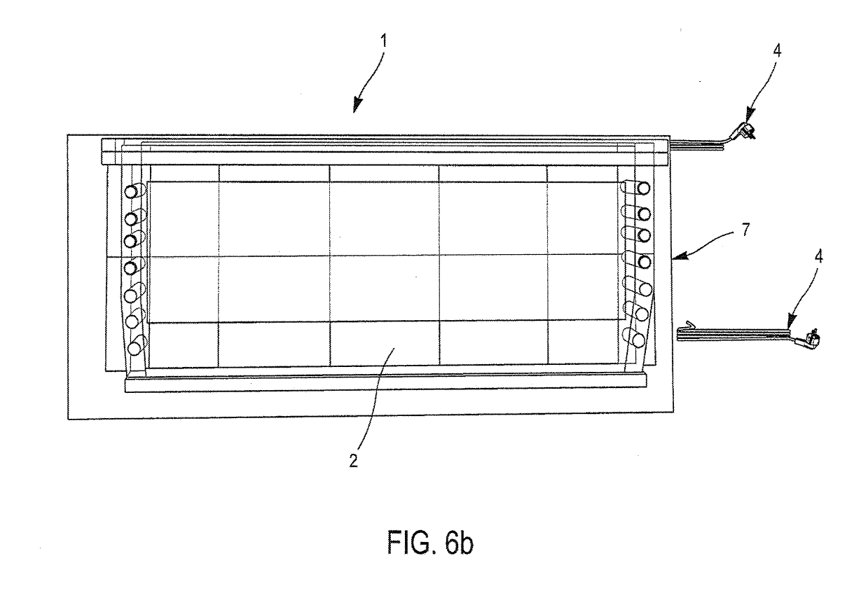

According to a second embodiment, the photobioreactor is parallelepipedic (FIGS. 6). Photovoltaic cells (2) are arranged on both faces of one or a plurality of metal panels (7). The dimensions of these panels are those of the photobioreactor. These panels (X) are placed outside the photobioreactor, preferably between two stacked culture enclosures. The panels are connected to current injection contacts (8). The photovoltaic cells are electrically insulated from the metal panel by an insulator having good thermal conductivity such as Mylar®.

1. Photobioreactor for cultivating photosynthetic micro-organisms, comprising:
at least one culture enclosure for containing a culture medium of the micro-organisms;
reverse emission photovoltaic cells isolated from the culture medium, made of a direct gap material, and configured to emit light to the culture medium,
wherein said reverse emission photovoltaic cells include current injection contacts for receiving electric current which is used by said reverse emission photovoltaic cells to emit light.

2. Photobioreactor according to claim 1, wherein the reverse emission photovoltaic cells are arranged on panels.

3. Photobioreactor according to claim 1, wherein the reverse emission photovoltaic cells are cells with one or two junctions.

4. Photobioreactor according to claim 1, wherein the reverse emission photovoltaic cells are made of a III/V material.

5. Photobioreactor according to claim 1, wherein the reverse emission photovoltaic cells are placed in sealed containers of adapted transparency (TA) immersed in the culture medium.

6. Photobioreactor according to claim 1, wherein the photovoltaic cells are placed outside the culture enclosure(s), at a short distance from the external wall of the culture enclosure(s) and the external wall of the culture enclosure(s) consists of a material of adapted transparency for the passage of the wavelength(s) emitted by said photovoltaic cells.

7. Photobioreactor according to claim 6, comprising a plurality of parallelepipedic culture enclosures, stacked and separated by panels of photovoltaic cells.

8. Photobioreactor according to claim 1, comprising a system for cooling the photovoltaic cells.

9. Photobioreactor according to claim 1, comprising a system for mixing the culture medium.

10. Photobioreactor according to claim 1, wherein further;
said culture enclosure for containing the micro-organism culture medium is cylindrical, and
said photovoltaic cells isolated from the culture medium cover panels, said panels extending along approximately the entire height of the culture enclosure, placed in sealed tube of adapted transparency immersed in the culture medium and arranged as a tube having a polygonal cross-section.

11. Photobioreactor according to claim 1, further comprising:
a plurality of parallelepipedic culture enclosures, stacked and separated by panels containing said photovoltaic cells,
said panels having the dimensions of one face of the culture enclosure.
